SEQUENCE LISTING (1 sequence)

SEQ ID NO: 1 - protein, 53 aa, Homo sapiens
NSDSECPLSHDGYCLHDGVCMYIEALDKYACNCVVGYIGERCQYRDLKWWELR

(19) United States
(12) Patent Application Publication
(10) Pub. No.: US 2017/0252411 A1
(43) Pub. Date: Sep. 7, 2017

(54) Compositions Derived from Platelet Lysates and Uses in Vascularization

(71) Applicant: Emory University, Atlanta, GA (US)

(72) Inventors: Luke Brewster, Decatur, GA (US); Scott Robinson, Ann Arbor, MI (US); Ian Copland, Atlanta, GA (US)

(21) Appl. No.: 15/449,067

(22) Filed: Mar. 3, 2017

Related U.S. Application Data
(60) Provisional application No. 62/303,066, filed on Mar. 3, 2016.

(51) Int. Cl. A61K 38/36; A61K 35/35; A61K 35/28

(52) U.S. Cl. CPC A61K 38/363; A61K 35/28; A61K 35/35

(57) ABSTRACT

This disclosure relates to compositions comprising a fibrin polymer produced by mixing platelet lysates with a minimal essential medium and thrombin. Typically, an optimized concentration of a calcium salt is utilized. In certain embodiments, the disclosure contemplates compositions comprising a fibrin polymer made by processes disclosed herein that contains cells, endothelial cells, stem cells, adipose tissue derived stem cell (ASC), and/or human mesenchymal stem cells (MSCs). In certain embodiments, the disclosure contemplates uses of composition disclosed herein containing cells, or cells replicated in compositions reported herein, to improve vascularizing, repairing, and healing of a tissue due to an ischemic condition or injury.

### CROSS REFERENCE TO RELATED APPLICATIONS

This application claims the benefit of U.S. Provisional Application No. 62/303,066 filed Mar. 3, 2016. The entirety of this application is hereby incorporated by reference for all purposes.

### STATEMENT REGARDING FEDERALLY FUNDED RESEARCH

This invention was made with government support under Rb01EB011566, UL1TR000454, and KO8HL119592 awarded by the NIH. The government has certain rights in the invention.

### INCORPORATION-BY-REFERENCE OF MATERIAL SUBMITTED S A TEXT FILE VIA THE OFFICE ELECTRONIC FILING SYSTEM (EFS-WEB)

The Sequence Listing associated with this application is provided in text format in lieu of a paper copy, and is hereby incorporated by reference into the specification. The name of the text file containing the Sequence Listing is 15101US_ST25.txt. The text file is 1 KB, was created on Mar. 3, 2017, and is being submitted electronically via EFS-Web.

### BACKGROUND

Peripheral arterial disease (PAD) is a common circulatory problem in which narrowed arteries reduce blood flow to the limbs. If PAD is caused by a buildup of plaques in blood vessels (atherosclerosis), there is also a risk of developing critical limb ischemia (CLI). CLI occurs when injuries or infections progress and cause tissue death (gangrene) sometimes requiring amputation of the affected limb. Surgical revascularization is another option to save the affected limb of a patient. Many CLI patients do not have revascularization options. Thus, there is a need to identify improved methods to promote vascular regeneration.

Liew et al. report potential for mesenchymal stem cell transplantation in critical limb ischemia. Stem Cell Res Ther. 2012; 3(4): 28.

In natural blood clotting processes, thrombin cleaves fibrinogen to form fibrin. Platelets contain fibrinogen. Walenda et al. report that a human platelet lysate gel provides a three dimensional-matrix for enhanced culture expansion of mesenchymal stromal cells. Tissue Eng Part C Methods, 2012, 18(12):924-34. See also Bouckenooghe al., Standardized and clinical grade human platelet lysate hydrogel (HPLG) for the optimization of large-scale expansion of human mesenchymal stem cells, Cytotherapy, Volume 17, Issue 6, S6; and Fortunato et al., Sci Rep. 2016; 6: 25326.

References cited herein are not an admission of prior art.

### SUMMARY

This disclosure relates to compositions comprising a fibrin polymer produced by mixing platelet lysates with a minimal essential medium and thrombin. Typically, an optimized concentration of a calcium salt is utilized. In certain embodiments, the disclosure contemplates compositions comprising a fibrin polymer made by processes disclosed herein that contains cells, endothelial cells, stem cells, adipose tissue derived stem cell (ASC), and/or human mesenchymal stem cells (MSCs). In certain embodiments, the disclosure contemplates uses of composition disclosed herein containing cells, or cells replicated in compositions reported herein, to improve vascularizing, repairing, and healing of a tissue due to an ischemic condition or injury.

In certain embodiments, an activating solution is prepared containing a minimal essential medium, thrombin, and a calcium salt at desirable concentrations for production of a hydrogel containing a fibrin polymer formed from fibrinogen derived from platelet lysates. In certain embodiments, the disclosure contemplates the additions of exogenously added growth factors such as EGF, bFGF, PDGF-BB, compounds reported in FIG. 10A, and combinations thereof.

In certain embodiments, the disclosure contemplates a composition having about a 1:1, or 1:1.5, or 1.5:1, or 1:2, or 2:1, or between a 1:1 and a 1.5:1 mixture, or between a 1.5:1 and a 1:1 mixture or between a 1:1 and a 2:1 mixture, or between a 2:1 and a 1:1 by volume mixture of platelet lysates and a liquid minimal essential medium respectively wherein the minimal essential medium contains desirable concentrations of a calcium salt and thrombin to produce the fibrin polymer.

In certain embodiments, the disclosure presents a procedure for culturing cells, such as endothelial cells, stem cells, ASCs and MSCs. In certain embodiments, the cells are cultured under standard conditions with a minimum essential medium optionally containing nucleosides, L-glutamine, and serum supplementation with fetal bovine serum.

In certain embodiments, the disclosure contemplates a mixture as reported herein to produce a fibrin polymer, where the fibrin content in the composition is about 225 micro grams/mL, or between 220 and 230 micro grams/mL, or between 210 and 240 micro grams/mL, or between 200 and 250 micro grams/mL, or between 150 and 300 micro grams/mL. In certain embodiments, the concentration of the calcium salt in the composition is about 5 mM, or the concentration of the calcium salt in the composition is above 1.8 mM or the calcium salt is more than, about or between 2 mM and 10 mM, or the calcium salt is more than or about 3, 4, 5, 6, or 7 mM.

In certain embodiments, the concentration of thrombin in the composition is about 2 U/mL, or the concentration of thrombin in the composition is more than or between 0.1 U/mL and 3 U/mL.

In certain embodiments, the platelet lysate is derived from platelets in platelet rich plasma.

In certain embodiments, the platelets are lysed by freezing and thawing the platelet at a temperature about or below zero, negative 25 or negative 60 degrees Celsius or lower. In certain embodiments, the thawed lysed platelets are centrifuged and filtered. In certain embodiments, thawed lysed platelets are centrifuged at about or at least 1500g for about or at least 2, 5, or 10 min. In certain embodiments, lysed platelets are thawed at about or at least 25, 30, 35, or 37 Celsius. In certain embodiments, thawed lysed platelets are centrifuged at about or at least 5,000 g, 7,500 g, or 10,000 g for about or at least 2, 5, or 10 min and filtered.

In certain embodiments, the disclosure contemplates a method of lysing platelets using a two sequential freeze/thaw cycles, wherein the first freezing is at negative 80° C. for at least 48 h, rapidly thawing at 37° C. for at least 8 h.

In certain embodiments, the minimal essential media contains about the concentrations of components as provided in table 1 having plus or minus 10%, 20%, 30%, 40%, or 50% of the concentration of each of the individual compounds, salts, amino acids, vitamins, saccharides, or nucleosides.

In certain embodiments, the disclosure presents evidence that under appropriate culture conditions, fibrin polymers can spontaneously form MSC spheroids. In certain embodiments, the disclosure contemplates the formation of spheroids from the sprouting of cells.

In certain embodiments, the disclosure contemplates methods of treating or preventing tissue necrosis due to ischemia comprising administering or implanting a composition disclosed herein to subject in need thereof.

In certain embodiments, the disclosure contemplates recruitment of remote cells, such as endothelial cells, human umbilical vein endothelial cells (HUVECs) by fibrin polymer containing cells as reported herein. In certain embodiments, the disclosure contemplates migration of remote cells by fibrin polymer containing cells as reported herein. In certain embodiments, the disclosure contemplates a method of promoting cell retention and survival in desired tissue to promote vascular regeneration by angiogenesis and arteriogenesis. In certain embodiments, the fibrin polymer, when implanted into ischemic limbs, results in vascularization.

In certain embodiments, the disclosure contemplates the treatment of subject suffering from peripheral arterial disease and critical limb ischemia. In certain embodiments, the subject is diagnosed with diabetes, e.g., type I or type II. In certain embodiments, the disclosure contemplates administration or implantation of materials disclosed herein, e.g., cells replicated in compositions disclosed herein, e.g., containing MSC spheroids, into ischemic tissues in an effective amount to provide concentrated population of retained cells as reported herein at targeted delivery sites.

In certain embodiments, this disclosure relates to methods of treating or preventing diseases and conditions reported herein or promoting vascularization in a tissue comprising administering or implanting cells, endothelial cells, stem cells, adipose tissue derived stem cell (ASC), and/or human mesenchymal stem cells (MSCs) replicated in a compositions disclosed herein in an effective amount to a subject in need thereof. In certain embodiments, the cells are obtained from a subject with diabetes, cardiovascular disease, subject over 55, 60, 65, years old, or combinations thereof. In certain embodiments, the cells are in the form of a spheroid produced in compositions using processes disclosed herein.

### BRIEF DESCRIPTION OF THE DRAWINGS

FIG. 1A illustrates PL hydrogels self-assembled with thrombin activation. Fibrinogen rich platelet lysate (PL) was generated by exposing human PL to sequential rounds of freeze thaw cycles with a rapid warming phase. Immediately prior to hydrogel formation, frozen PL aliquots were rapidly warmed to 37° C., centrifuged and filtered. The addition of thrombin led to self-assembly of 3D hydrogels.

FIG. 1B shows data from scanning electron microscopy performed on 50% PL and 1.0 mg/mL and 2.5 mg/mL fibrin hydrogels. Representative images are shown for each condition at 20,000×. Scale bar=1 micrometer. Despite a fibrin concentration of 0.250 mg/mL, PL gel has an intermediary appearance between the 1 and 2.5 mg/mL fibrin gels in both confocal and electron microscopy.

FIG. 2A shows the structural properties of PL hydrogels. 70 kDa FITC-dextran was incorporated into 50% PL fibrin hydrogels. Percent of FITC-dextran release was quantified over 20 days in the absence of aprotinin.

FIG. 2B shows the structural properties of PL hydrogels. 70 kDa FITC-dextran was incorporated into 50% PL fibrin hydrogels. Percent of FITC-dextran release was quantified over 20 days in the presence of aprotinin. Here PL gel had sustained release of dextran over 20 days that was superior over the fibrin gels. Aprotinin abrogated this benefit. Alexafluor-488 conjugated fibrinogen was incorporated in 50% PL and fibrin hydrogels.

FIG. 2C presents data on scaffold degradation analyzed by quantifying release of labeled fibrinogen over 7 days in the absence of aprotinin.

FIG. 2D presents data on scaffold degradation analyzed by quantifying release of labeled fibrinogen over 7 days in the presence of aprotinin. Here PL gel had superior integrity over fibrin gels that persisted, although to a lesser degree, in the presence of aprotinin.

FIG. 2E presents data on oscillatory rheology used to assess mechanical properties of the 50% PL and fibrin hydrogels storage modulus calculated from an average G′ or G″ at 0.5% strain over a frequency sweep from 0.01 to 1 Hz. Total protein released from 50% PL hydrogels over 20 days was calculated using a modified Bradford assay over time in the presence and absence of aprotinin. Protein release is shown both as cumulative protein released. G′=Storage modulus

FIG. 2F presents data on oscillatory rheology used to assess mechanical properties of the 50% PL and fibrin hydrogels loss modulus calculated from an average G′ or G″ at 0.5% strain over a frequency sweep from 0.01 to 1 Hz. G″=Loss modulus.

FIG. 2G shows data on the total protein released from 50% PL hydrogels over 20 days calculated using a modified Bradford assay over time in the presence and absence of aprotinin. Protein release is shown both as cumulative protein released.

FIG. 2H shows data on the total protein released from 50% PL hydrogels over 20 days calculated using a modified Bradford assay over time in the presence and absence of aprotinin. Protein release is shown as the percent of total protein released from the hydrogels

FIG. 2I shows data on PDGF-BB released from 50% PL hydrogels over 20 days measured with ELISA in the presence and absence of aprotinin. Total PDGF-BB release is shown as cumulative protein.

FIG. 2J shows data on PDGF-BB released from 50% PL hydrogels over 20 days measured with ELISA in the presence and absence of aprotinin. Total PDGF-BB release is shown as percent of total protein released from the hydrogels. Aprotinin was not necessary to improve gel integrity or to sustain the protein release or PDGF-BB release in PL gels.

FIG. 3A shows data on the average cell invasion length quantified over time in hydrogels cultured under serum free conditions. Total cell invasion was significantly improved in the PL gel compared to both fibrin gels. Cell pellets containing MSCs and HUVECs labeled with PKH26 were embedded in PL and fibrin hydrogels. Representative bright field images of cell pellets within different scaffolds at 3 days captured combined MSC and EC invasion.

FIG. 3B presents representative fluorescent images of HUVEC sprouting from co-culture at 3 days. Average EC sprout length was quantified over time. Here MSC's angiogenic and stromal activity on ECs was significantly greater in PL gel than in the 2.5 mg/mL fibrin gel. Endothelial specific sprouting determined using fluorescent microscopy to capture only the PKH26 labeled HUVECs specifically from the co-culture assay.

FIG. 4A shows the effect of PL scaffold on MSC and HUVEC proliferation. Proliferation of MSCs in PL and fibrin gels determined by MTS assay. Groups were normalized to MSCs grown in a monolayer under serum free conditions.

FIG. 4B shows the effect of PL scaffold on MSC and HUVEC proliferation. Proliferation of HUVECs in PL and fibrin gels. All groups are normalized to HUVECS grown in a monolayer under serum free conditions.

FIG. 5A shows data on MSC and EC invasion of PL scaffolds. Cell pellets containing single cell type of either MSCs or HUVECs embedded in PL and fibrin hydrogels. Average invasion length from MSC pellets over 3 days. Cell pellets containing single cell type of either MSCs or HUVECs embedded in PL and fibrin hydrogels. Representative images of MSC sprout formation in PL, low and high concentration fibrin hydrogels at 3 days.

FIG. 5B shows data MSC and EC invasion of PL scaffolds. Cell pellets containing single cell type of either MSCs or HUVECs embedded in PL and fibrin hydrogels. Average sprout length from HUVEC pellets over 3 days. Cell pellets containing single cell type of either MSCs or HUVECs embedded in PL and fibrin hydrogels. Representative images of HUVEC sprout formation in PL, low and high concentration fibrin hydrogels at 3 days.

FIG. 6A shows that PL hydrogel recruits remote endothelial cells. Cell free PL promotes migration of HUVECs when compared to fibrin only in a transwell migration assay.

FIG. 6B shows that PL hydrogel recruits remote endothelial cells. MSCs embedded in PL promote migration of HUVECs when compared to MSCs embedded in fibrin hydrogels.

FIG. 7A shows data on MSCs in PL Gel Restore Perfusion Rapidly in NOD-SCID mice treated with MSCs in PL gel compared to control groups (saline and PL gel alone, and MSCs in saline) indicating limb perfusion in each group at 1 and 8 days. Quantification of limb perfusion at 1 and 8 days in the ischemic are of the leg pertaining to the calf muscle. Here the ischemic right leg perfusion normalizes (right to left ratio of 1) in the PL+MSCs group by day 8 after HLI. Comparison of PL+MSCs to all groups was significant.

FIG. 7B shows data on MSCs in PL Gel Restore Perfusion Rapidly in NOD-SCID mice treated with MSCs in PL gel compared to control groups (saline and PL gel alone, and MSCs in saline). Quantification of limb perfusion of the ischemic leg including the foot showed a significant difference between the PL+MSCs and saline only group.

FIG. 8 illustrates the preparation of a MSC spheroid with sprout formations and implantation for the promotion of neovascularization.

FIG. 9 shows data on the improvement of MSC survival and MSC delivery with PL gel vs. saline.

FIG. 10A show experiments on the contents of MSCs for detection of growth factors with antibodies to the growth factors or other molecules. There are challenges using MSCs derived from diabetic patients for peripheral arterial disease because diabetic MSCs do not expand or survive normally, do not secrete EGF. Diabetic MSCs and exogenous EGF regain AKT but not ERK signaling.

FIG. 10B shows data on PL media and FBS Media as outlined in 10A. The arrow indicates the area for EGF antibody. The pound sign (#) for b-FGF. The asterisk (*) for PDGF-BB.

FIG. 10C shows data on the detection of growth factors in MSCs grown in PL verses FBS as outlined in 10A indicating an increase in EGF in MSCs grown in PL.

FIG. 10D shows data for diabetic MSCs as outlined in 10A indicating an increase in EGF.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise.

As used herein, “subject” refers to any animal, preferably a human patient, livestock, or domestic pet.

As used herein, the terms “prevent” and “preventing” include the prevention of the recurrence, spread or onset. It is not intended that the present disclosure be limited to complete prevention. In some embodiments, the onset is delayed, or the severity is reduced.

As used herein, the terms “treat” and “treating” are not limited to the case where the subject (e.g. patient) is cured and the disease is eradicated. Rather, embodiments of the present disclosure also contemplate treatment that merely reduces symptoms, and/or delays disease progression.

The term “mesenchymal stromal cells” refers to the subpopulation of fibroblast or fibroblast-like nonhematopoietic cells with properties of plastic adherence and capable of in vitro differentiation into cells of mesodermal origin. Mesenchymal stromal cells be derived from bone marrow, adipose tissue, umbilical cord (Wharton's jelly), umbilical cord perivascular cells, umbilical cord blood, amniotic fluid, placenta, skin, dental pulp, breast milk, and synovial membrane. Mesenchymal stromal cells have a clonogenic capacity and can differentiate into several cells of mesodermal origin, such as adipocytes, osteoblasts, chondrocytes, skeletal myocytes, or visceral stromal cells. The term, “mesenchymal stem cells” refers to the cultured (self-renewed) progeny of primary mesenchymal stromal cell populations. Mesenchymal stromal/stem cells (MSCs) refers to mesenchymal stromal and/or mesenchymal stem cells.

Bone marrow derived mesenchymal stromal cells are typically expanded ex vivo from bone marrow aspirates to confluence. Certain mesenchymal stromal/stem cells (MSCs) share a similar set of core markers and properties. Typically mesenchymal stromal/stem cells (MSCs) may be defined as positive for CD105, CD73, and CD90 and negative for CD45, CD34, and have the ability to adhere to plastic. See Dominici et al. Minimal criteria for defining multipotent mesenchymal stromal cells. The International Society for Cellular Therapy position statement. Cytotherapy. 2006, 8(4):315-7.

Adipose tissue-derived stem cells (ADSCs) are multipotent, undifferentiated, self-renewing progenitor cell population isolated from adipose tissue. One method to isolate ADSCs from fat tissue relies on a collagenase digestion, followed by centrifugal density gradient separation. In vitro, ADSCs typically display a spindle-shaped morphology and lack the intracellular lipid droplets as seen in adipocytes. Isolated ADSCs are typically expanded in monolayer cultures with a growth medium containing fetal bovine serum and/or human platelet lysate. ADSCs have the stem cell-specific surface markers, such as CD90, CD105, CD73, and lack the expression of the hematopoietic markers CD45 and CD34.

As used herein the term, the term “platelet lysate” refers to the products of platelet lysis. Platelets (thrombocytes) are typically collected in pooled whole blood-derived buffy coats. When whole blood is centrifuged, cells are separated in density layers. Plasma will be on the top. Red blood cells on the bottom. A buffy coat, which contains platelets and white blood cells (leukocytes), sits in between the plasma and red blood cells.

Once collected, multiple buffy coat units (e.g., 4) may be mixed with a plasma layer and centrifuged again at a lower spin rate, e.g., ten-fold. The resulting top layer is referred to as “platelet rich plasma.” In certain embodiments, the disclosure contemplates using platelet rich plasma wherein the platelet concentration is about or above 0.5 to 2.0×10⁹ platelets per mL or 1.0 to 2.0×10⁹ platelets per mL or 1.2 to 1.8×10⁹ platelets per mL. In certain embodiments, the platelet concentration is about or above 1.5×10⁹/mL.

The process of purifying platelets may be automated using an apheresis machine. Inside an apheresis machine there is a blood chamber that centrifuges the blood, separating it into layers. Red cells are heaviest and sit at the bottom, platelets and white cells are in the middle (buffy coat) Plasma is at the top as it is the lightest.

The isolation of purified platelets from human blood buffy coat can also be done by centrifugation of over an iodixanol density barrier of 1.063 g/ml (350 g for 15 min at 20 degrees Celsius). Platelets will be below the plasma. The bottom of the platelet band will show an increase in concentration of contaminant leukocytes and erythrocytes.

In certain embodiments, lysing plasma cells is by freezing and thawing. However, other methods are contemplated. Mechanical lysis, the use of shear forces, and sonication are contemplated for producing lysate. Lysis buffer, typically by placing the cells in a hypotonic solution, are yet another option. The lysis process may consist of combinations of these methods.

A “minimal essential medium” refers a medium containing salts of calcium, magnesium, potassium, sodium, phosphate, and bicarbonate, vitamins, and essential amino acids. The 12 essential amino acids are: L-arginine; L-cystine; L-glutamine; L-histidine; L-isoleucine; L-leucine; L-methionine; L-phenylalanine; L-threonine; L-tryptophan; L-tyrosine; and L-valine. An MEM is often supplemented with components such as bicarbonate or glutamine. In certain embodiments, this disclosure contemplates a minimal essential medium supplemented with non-essential amino acids: L-ala; L-asn; L-asp; L-glu; L-gly; L-pro and L-ser. In certain embodiments, this disclosure contemplates a minimal essential medium supplemented with nucleosides (ribonucleosides and/or deoxyribonucleosides). In certain embodiments, this disclosure contemplates a minimal essential medium supplemented with L-glutamine, e.g., at least 0.292 gm/L of L-glutamine. In certain embodiments, this disclosure contemplates a minimal essential medium supplemented with sodium bicarbonate, e.g., with at least 0.35 gm/L or 2.2 gm/L of sodium bicarbonate.

As used herein, “thrombin” refers to the human or non-human serine protease that cleaves fibrinogen at Arg residues resulting in the formation of fibrin. Thrombin is a two chain enzyme composed of an N-terminal “A” chain and a C-terminal “B” chain which are covalently bound through a disulfide bond. Human thrombin is 13 amino acids shorter than the bovine thrombin due to a thrombin cleavage site on the human protein that is not present in the bovine protein. The activity of thrombin is expressed in NIH units which can be obtained by direct comparison to a NIH Thrombin Reference Standard, Lot K. The specific activity of commercially available thrombin is typically about 3800 NIH units/mg.

Epidermal growth factor (EGF) is a growth factor that stimulates cell growth, proliferation, and differentiation by binding to its receptor EGFR. The EGF precursor is believed to exist as a membrane-bound molecule which is proteolytically cleaved to generate the acid peptide hormone that stimulates cells to divide. EGF stimulates the growth of various epidermal and epithelial tissues in vivo and in vitro and of some fibroblasts in cell culture. Human recombinant EGF is sold having (SEQ ID NO: 1) NSDSECPLSHDGYCLHDGVCMYIEALDKYACNCVV GYIGERCQYRDLKWWELR.

Basic fibroblast growth factor is also known as, bFGF FGF-basic, FGF-β, FGF2, or heparin binding growth factor. Basic FGF has two sequences that are characteristic of heparin-binding domains. However, synthetic peptides including flanking sequences also bind to heparin.

Platelet-derived growth factor (PDGF) is a dimeric glycoprotein composed of two A (−AA) or two B (−BB) chains or a combination of the two (−AB). PDGF is synthesized, stored (in the alpha granules), and released by platelets upon activation. PDGF is a growth factors and has a role in blood vessel formation (angiogenesis).

### Ischemia

In certain embodiments, this disclosure relates to methods of treating or preventing ischemia comprising administering or implanting a composition disclosed herein to subject in need thereof. In certain embodiments, the subject is at risk of, exhibiting symptoms of, or diagnosed with peripheral arterial disease (PAD) or critical limb ischemia (CLI).

Ischemia refers to a vascular condition involving an interruption in the blood supply to a tissue, organ, or extremity that may lead to tissue death. Oxygen is carried to tissues in the blood. Insufficient blood supply causes tissue to become starved of oxygen. Without intervention, ischemia may progress to tissue necrosis and gangrene. Paralysis is a sign of acute arterial ischemia and signals the death of nerves supplying the extremity. Foot drop may occur as a result of nerve damage. Because nerves are sensitive to hypoxia, limb paralysis or ischemic neuropathy may persist after revascularization and may be permanent.

Ischemia can be caused by embolism, thrombosis of an atherosclerotic artery, or trauma. Venous problems like venous outflow obstruction and low-flow states can cause acute arterial ischemia. An aneurysm is one of the most frequent causes of acute arterial ischemia. Other causes are heart conditions including myocardial infarction, mitral valve disease, chronic atrial fibrillation, cardiomyopathies, and prosthesis, in all of which thrombi are prone to develop. Frostbite may be a cause if ischemic injury.

Peripheral artery disease (PAD) refers to thickening of an arteries lining typically caused by a build-up of plaque (atherosclerosis). Atherosclerosis narrows or blocks blood flow, reducing circulation of blood to the legs, feet, or hands. Critical limb ischemia is the advanced stage of peripheral artery disease (PAD). Critical Limb Ischemia (CLI) refers to obstruction of the arteries which markedly reduces blood flow to the extremities (hands, feet and legs) and has progressed to the point of resting pain. Other signs of CLI include skin ulcers, sores, or gangrene.

In certain embodiments, this disclosure contemplates using methods disclosed herein in combination with other treatments of ischemic conditions.

Treatment options include administration of an anticoagulant, thrombolysis, embolectomy, surgical revascularisation, or amputation. Anticoagulant therapy may be initiated to prevent further enlargement of the thrombus. Continuous IV unfractionated heparin is a treatment option. If the ischemic condition of a limb is stabilized with anticoagulation, emboli may be treated with catheter-directed thrombolysis using intra-arterial infusion of a thrombolytic agent (e.g., recombinant tissue plasminogen activator (tPA), streptokinase, or urokinase). A percutaneous catheter is typically inserted into the femoral artery and threaded to the site of the clot to infuse the drug. Direct arteriotomy may be used to remove a clot. Surgical revascularization may be used in the setting of trauma (e.g., laceration of the artery).

Platelet Lysate (PL) Hydrogel for Endothelial Cell and Mesenchymal Stem Cell-Directed Neovascularization

PAD is a common circulatory problem in which narrowed arteries reduce blood flow to the limbs. If PAD is caused by a buildup of plaques in blood vessels (atherosclerosis), there is also a risk of developing critical limb ischemia (CLI). This condition begins as open sores that don't heal, an injury, or an infection of the feet or legs. CLI occurs when such injuries or infections progress and cause tissue death (gangrene), sometimes requiring amputation of the affected limb.

Surgical revascularization is another option to save the affected limb of a patient compared to primary amputation, but up to half of all CLI patients do not have revascularization options. Thus novel approaches to promote vascular regeneration, such as cellular therapy, are needed.

Mesenchymal stem cells (MSCs) are a desirable cell type for vascular regeneration via angiogenesis and arteriogenesis. The proangiogenic effects of MSCs are largely due to their paracrine activity on surrounding cells. Nevertheless, despite promising early results, the effectiveness of cellular therapies in preventing amputations remains to be proven. This is due, in part, to poor MSC retention and survival in the desired tissue.

A hydrogel (scaffold) has been designed to improve MSC retention, survival, and engraftment into the desired tissue beds. This gel is derived from pooled human platelet lysate (PL), which is utilized in a soluble form as a supplement, has improved abilities to expand MSCs in culture and avoids contamination by fetal bovine serum, supplement from non-human species. The structural properties of this scaffold and have further confirmed pro-angiogenic effects of PL: labelled cell pellets containing MSC and Huvec cells were embedded in PL or Fibrin hydrogel for comparison.

PL promotes MSC proliferation and hydrogel invasion by formation of spheroids. Spheroids represent a 3D cell culture format, which can mimic cell behaviors, closely, similar to physiological architectures compared to monolayer 2D tissue cultures in terms of cell morphology and cell functionality.

The ability of PL to rapidly form spontaneous MSC spheroids under appropriate culture conditions is a novel property of this hydrogel. MSC encapsulated PL spheroids provide an alternative strategy for cell delivery: MSC spheroids could be directly injected into the ischemic tissues to provide a concentrated population of retained MSCs at targeted delivery sites. Alternatively, the spheroids could be dispersed within an additional hydrogel matrix along the desired path of neovascularization. The production of MSC spheroids with PL preserves cell viability and function, indicating that MSC's within the spheroids retain their regenerative capacity.

Experiments described herein provide that a specific platelet lysate (PL) hydrogel has many of the desired characteristics to promote MSC retention and survival for cellular therapy including a favorable gel density, sustained gel integrity, and a superior growth factor retention and release profile. In vitro, PL gel promotes MSC invasion and stimulation of EC sprouting. The proangiogenic effect of a MSC seeded PL scaffold was demonstrated in vivo, where PL gel with human MSCs rapidly (8 days) regenerated vascular perfusion in a NOD-SCID HLI model.

The use of MSCs in PL gel is an exciting MSC delivery strategy for targeted vascular regeneration. In its soluble form, PL increases the expansion capacity of MSCs. PL is superior to bovine serum for culture of MSCs and can be derived from cross-matched blood products to reduce the risk of immunogenicity in cell therapy. Also, it avoids the use of xenogenic cell culture supplements and subsequent xenogenic contamination. In its hydrogel form, PL can be used as a cell delivery vehicle that also retains the nutritive functions of soluble PL, thereby enabling MSC survival and engraftment. This may be particularly important to clinical outcomes when using cells from patients with impaired or depleted stem cell populations, such as those with coronary artery disease, stroke, diabetes mellitus, and tobacco smokers. Overcoming impaired stem cell function is important for patients with CLI as they commonly have several risk factors.

MSCs from CLI patients cultured in PL-supplemented media retain angiogenic activity. In these patients, the use of soluble PL is helpful to expand MSCs in a clinically useful timeframe. The biologic effects of PL gel allow for sustained nutritive support after delivery in vivo. The number of functioning cells in the desired tissue appears to be important for therapeutic impact of cell therapy, therefore it is important that cell delivery strategies overcome poor cell retention and viability.

The effect on MSC invasion in PL hydrogels disclosed herein were tested in an austere serum free environment. MSCs in PL hydrogels had significantly greater invasion than in fibrin gels. The PL scaffold has unique structural properties that allow it to behave as a viscoelastic solid with improved mechanical properties compared to fibrin only hydrogels. The relatively soft nature of PL gel likely contributes to the favorable MSC invasion and endothelial sprouting.

MSC growth and proliferation appears to be is regulated by the growth factor milieu contained within the PL scaffold. The PL scaffold led to superior invasion of HUVECs compared to both high and low fibrin controls. Data indicate that PL gels contain a targeted growth factor milieu that induces sprouting of ECs with minimal effect on mitogenic activity. MSCs in PL gels had increased invasion and proliferation. The PL gels native form embedded with MSCs has the ability to recruit remote endothelial cells as demonstrated in the transwell migration assay. PL gels embedded with MSCs recruits host ECs for neovascularization following implantation in ischemic tissues. The coordinated neovascular activity of PL gels and MSCs was further supported in vivo, where rapid and complete neovascularization occurred in 8 days, which was increased significantly when compared to PL gels alone and MSCs alone. This combinatorial benefit enables single cell type (MSC) delivery in the PL hydrogels, which greatly simplifies the regulatory barriers to clinical translation.

Differences between the 1 and 2.5 mg/mL fibrin gels were identified. Here the 1 mg/mL fibrin gels acted similar to PL gels in both mechanical and biologic testing. Taken together, these data suggest that the mechanical properties of the PL hydrogels (i.e. stiffness, porosity) selectively promote MSC invasion, while the growth factor milieu contained within the PL promotes MSC over EC proliferation. Mechanically, the PL scaffold behaved as a viscoelastic solid. An organized network of fluorescently labeled fibrin was clearly visible within in the PL scaffold, and the storage and loss modulus of PL were comparable to that of the 1 mg/mL fibrin gels, which had four times the fibrin content. SEM imaging of the PL hydrogels revealed a microstructure distinct from that seen in the fibrin gels, indicating that other structural components are likely involved in PL gel formation and its favorable growth factor release kinetics.

Composite structures with fibrin and additional components such as collagen can improve the mechanical properties of hydrogels without increasing protein content. The superior performance of PL may be the result of reaction conditions distinct from fibrin-only gels during thrombin induced polymerization. Although it is not intended that embodiments of this disclosure be limited by any particular mechanism, macromolecules within the PL may alter fibrin binding sites or serve as molecular crowders, improving the structural properties of the scaffold. The presence of an enriched milieu of proteins within PL, including Factor XIII, also lead to enhanced fibrin crosslinking and strengthening of the PL gel. Additionally, extracellular proteins (i.e. fibronectin, collagen), proteoglycans, and adhesion proteins such as Von Willebrand Factor within the PL may incorporate into and reinforce the fibrin network as well as provide sustained release of PL's robust growth factors and angio genic compounds.

PL gels provided sustained release of endogenous PDGF-BB over 20 days in vitro, and 45% of PDGF-BB was still present in the PL gels at that distant time point. This excellent retention is far superior to that seen in optimized formulations of fibrin-only hydrogels in vitro, whereby there is approximately 6% of growth factor retained after 7 days. The desirable sustained release of growth factors from PL hydrogels identified here may be due to sequestration by the microstructural networks as well as the result of resistance of PL to autolysis compared to fibrin. Specific angiogenic compounds in PL include PDGF, VEGF, EGF, and BDNF. These angiogenic compounds may contribute to PLs' superior performance augmenting neovessel formation compared to fibrin gel.

In addition to serving as a proangiogenic growth factor, PDGF-BB is a mediator of MSC engraftment into tissue. Thus delivery of MSCs in PL gel improves MSC therapy by both providing a scaffold with nutritive growth factors and sustained delivery of these growth factors over time.

Through standard cross matching of blood products, one can substantially reduce the risk of a deleterious immunogenic response to PL while allowing for a more reproducible clinical effect.

In certain embodiments, this discloser contemplates PL supplementation for MSC expansion in addition to the PL gel delivery system. Rapid and complete neovascularization of NOD-SCID mice was found after HLI with human MSCs in PL gel, and significantly improved neovascularization of ischemic tissue compared to control groups.

In vitro fibrin gels were tested in two different concentrations. The higher concentration represents a physiologically relevant fibrinogen level representative of normal human serum that more precisely mimicked the micro network of PL gel, while the lower concentration fibrin gels mimicked the PL gel's actual fibrin concentration. These two fibrin gels had different effects on the MSC and EC behaviors in the gel with PL gel and the 1 mg/mL fibrin gel being most similar.

The use of a PL scaffold for delivery of MSCs in a setting of therapeutic angiogenesis offers several advantages over existing cell therapy platforms. PL is derived entirely from human blood products, and therefore it does not pose a risk for xenogenic contamination. PL enables delivery of autologous cells with a scaffold that could be cross-matched to further reduce the potential immunogenic risk of cell therapy. PL can provide both sustained delivery of growth factors and structural support for localized delivery of MSCs to desired tissue beds. The combination of these properties may be helpful in overcoming current clinical limitations to cell therapy.

The robust proliferative response of MSCs to the PL hydrogel also permits in vivo expansion of MSCs, thus enabling the use of a lower initial cell dose to achieve a therapeutic benefit. In vivo testing of MSCs in PL gel demonstrated robust and complete neovascularization by day 8 in a NOD-SCID HLI model.

### Production of Fibrinogen-Rich Platelet Lysate Hydrogels

Two units of expired human platelets were obtained from the Emory University blood bank through an IRB approved research protocol. The platelets were pooled and exposed to two sequential freeze/thaw cycles [freezing at 80° C. for 48 h, rapidly thawing at 37° C. for 8 h] followed by centrifugation at 1500 g for 10 min. The supernatant was collected and stored at 20° C. Prior to use, the platelet lysate was thawed at 37° C., centrifuged at 10,000 g for 10 min in 1.5 mL microcentrifuge tubes, and sequentially filtered through 0.45 and 0.2-micro-m syringe tip filters. Fibrinogen content was determined using an ELISA kit for human fibrinogen (Molecular Innovations). For hydrogel production, an activating solution was prepared containing AlphaMEM (Corning), bovine thrombin (Sigma), and CaCl₂ (Sigma). Cells were suspended in AlphaMEM at pre-specified concentrations and added to the activating solution. Hydrogels were polymerized by adding PL to the activating solution in a 1:1 ratio with a final concentration of CaCl2 and thrombin at 5 mM and 2 U/mL in a 50% PL gel, respectively. For the control fibrin hydrogel, fibrinogen from human plasma (Sigma) was dissolved in AlphaMEM then mixed with activating solution [final fibrinogen concentration was 2.5 mg/mL and 1.0 mg/mL for high and low concentration fibrin gels, respectively]. The 2.5 mg/mL fibrin-only hydrogel was chosen as a control because it represents a physiologically relevant concentration of fibrinogen that is equivalent to that found in human plasma. Additionally, the use of 2.5 mg/ml fibrin gels has been used extensively in the hydrogel invasion assay. The 1.0 mg/mL fibrin gels were selected as an additional control to more closely represent the concentration of fibrinogen found in PL solution. The 1.0 mg/ml fibrin hydrogels were mechanically equivalent to the 50% PL hydrogels, and hydrogels containing less than 1.0 mg/mL fibrinogen either would not form hydrogels or formed hydrogels that lacked the durability to be utilized in the assays. The addition of CaCl₂ to fibrin-only hydrogels caused precipitation of calcium phosphate, so for generation of fibrin-only hydrogels an activating solution was prepared containing only thrombin in AlphaMEM (which contains of CaCl₂ at a concentration of 1.80 mM). To convert mg/L to mM divide the concentration in mg/L by the molecular weight of the sample. CaCl₂ of 200 mg/L in AlphaMEM, CaCl₂ MW is 110.98. AlphaMEM CaCl₂ concentration of about 1.8 mM.

TABLE 1
Component of Alpha Modification of Eagle's
Medium (AlphaMEM) Formulation in mg/L
(Corning ™)

| | |
|---|---|
| CaCl₂ (anhydrous) | 200.00 |
| KCl | 400.00 |
| MgSO4 (anhydrous) | 97.70 |
| NaCl | 6800.00 |
| NaH2PO4•H2O | 140.00 |
| NaHCO3 | 2200.00 |
| L-Alanine | 25.00 |
| L-Arginine•HCl | 126.40 |
| L-Asparagine•H2O | 50.00 |
| L-Aspartic acid | 30.00 |
| L-Cysteine•HCl•H2O | 100.00 |
| L-Cystine•2HCl | 31.20 |
| L-Glutamic acid | 75.00 |
| L-Glutamine | 292.00 |
| Glycine | 50.00 |
| L-Histidine•HCl•H2O | 41.90 |
| L-Isoleucine | 52.50 |
| L-Leucine | 52.50 |
| L-Lysine•HCl | 72.50 |
| L-Methionine | 15.00 |
| L-Phenylalanine | 32.50 |
| L-Proline | 40.00 |
| L-Serine | 25.00 |
| L-Threonine | 47.60 |
| L-Tryptophan | 10.00 |
| L-Tyrosine•2Na•2H2O | 51.90 |
| L-Valine | 46.80 |
| Ascorbic acid | 50.00 |
| Biotin | 0.10 |
| D-Calcium pantothenate | 1.00 |
| Choline chloride | 1.00 |
| Folic acid | 1.00 |
| i-Inositol | 2.00 |
| Nicotinamide | 1.00 |
| Pyridoxine•HCl | 1.00 |
| Riboflavin | 0.10 |
| Thiamine•HCl | 1.00 |
| Vitamin B12 | 1.36 |
| D-Glucose | 1000.00 |
| Lipoic acid | 0.20 |
| Phenol red, Na | 10.00 |
| Sodium pyruvate | 110.00 |
| Thymidine | 10.00 |
| Adenosine | 10.00 |
| Cytidine | 10.00 |
| Guanosine | 10.00 |
| Uridine (anhydrous) | 10.00 |
| 2′-Deoxyadenosine | 10.00 |
| 2′-Deoxycytidine•HCl | 11.00 |
| 2′-Deoxyguanosine | 10.00 |

TABLE 2
Specification of Alpha Modification of Eagle's
Medium (AlphaMEM) Formulation
(Corning ™)

| | |
|---|---|
| NaHCO₃ | 2.20 |
| Powder (g/L) | 29.33 |
| 7.5% Solution (mL/L) | |
| L-Glutamine | 292.00 |
| Powder (mg/L) | 10.00 |
| 200 mM Solution (mL/L) | |
| pH | 7.0 ± 0.5 |
| Osmolality (mOsm/kg) | 290 ± 30 |

PL Rapidly Self Assembles into 3D Hydrogels with a Dense Fibrin Network

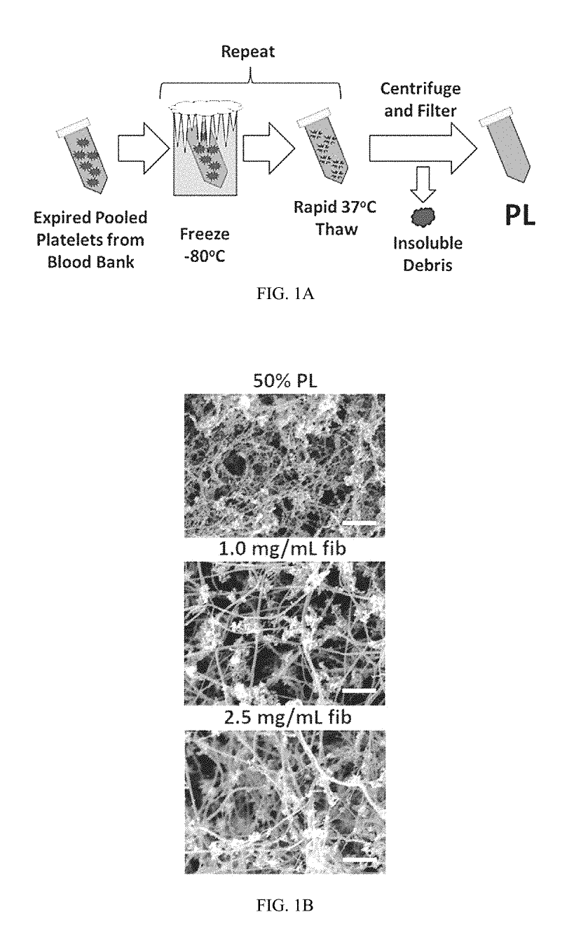

PL was generated by exposing human platelets to sequential freeze-thaw cycles with a rapid warming phase, which prevents the formation of cryoprecipitate (FIG. 1A). The fibrinogen concentration in the resultant PL solution determined with ELISA was 454 (±75) micrograms/mL. A solution of 50% PL was chosen for generation of our scaffold, so fibrin content in the gels was 225 micrograms/mL. The gel was polymerized by the addition of thrombin and calcium chloride to a 50% PL solution at 37 C. Confocal imaging of PL hydrogels loaded with 5% alexafluor-488 labeled fibrinogen revealed an organized fibrin network more dense than the 1.0 mg/mL fibrin comparison gel, and similar to that seen in control 2.5 mg/ml fibrin hydrogels. The labeled fibrinogen was incorporated randomly into the polymerizing fibrin fibers along with the native fibers. This was confirmed in a separate experiment as labeled fibrinogen alone did not form networks at low concentrations. Microstructural analysis with scanning electron microscopy revealed that the morphology of the PL consisted of thin, highly interconnected branched networks that were distinct from the fibrin hydrogels, which formed more elongated fibrils (FIG. 1B).

### PL Hydrogel Enables Sustained Release of Growth Factors

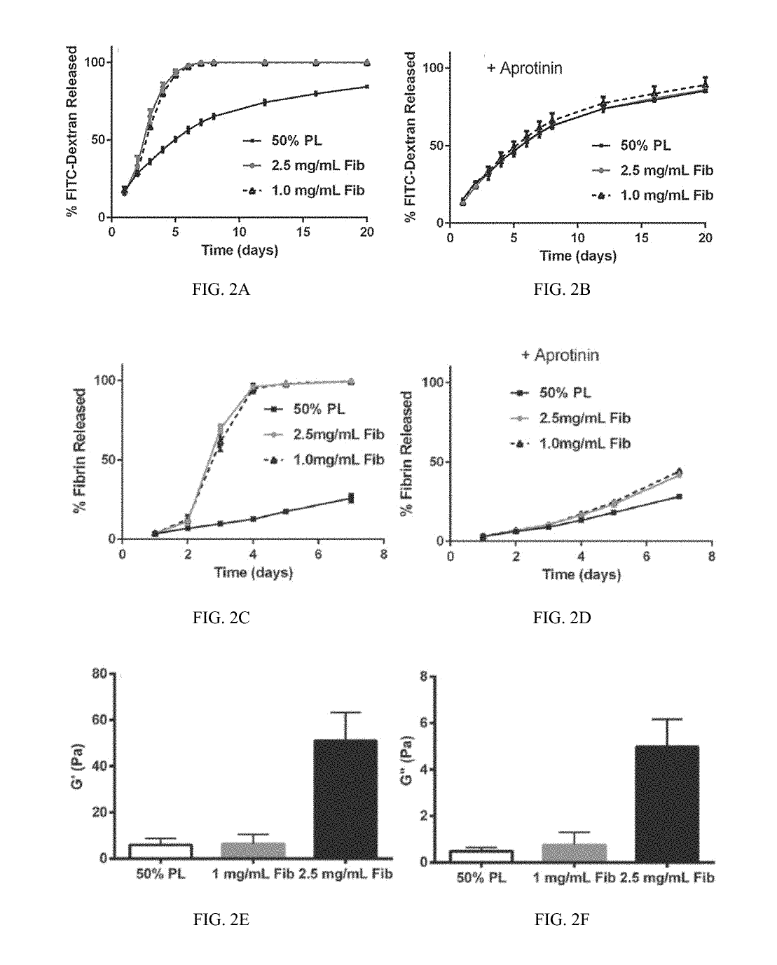

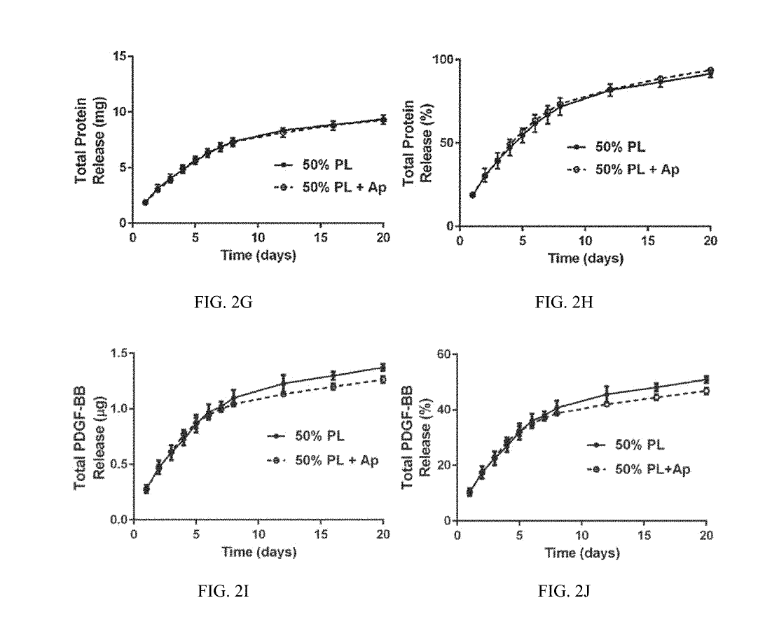

To examine the diffusion of soluble mediators from within the PL hydrogels, scaffolds were embedded with 70 kDa FITC-dextran. When incubated in PBS, 50% PL hydrogels had slow and sustained release of FITC-dextran over 20 days. Conversely, fibrin hydrogels rapidly released FITC-dextran from the scaffolds (FIG. 2A). The addition of the protease inhibitor aprotinin to PBS improved retention of FITC-dextran in the fibrin hydrogels but had no effect on the PL gel (FIG. 2B). To characterize the degradation of the PL scaffold, alexaflour-488 labeled fibrinogen was incorporated directly into PL and fibrin hydrogels. The hydrogels were incubated in PBS and the amount of fluorescent fibrin released into the PBS was quantified over 7 days. The fibrin hydrogels rapidly degraded after 48 h, but the PL hydrogels retained over 70% of the labeled fibrin over this time (FIG. 2C). The addition of aprotinin delayed degradation of the fibrin hydrogels, but the release of incorporated fibrin was still lower in the PL group (FIG. 2D). To evaluate the mechanical properties of the PL scaffold, oscillatory rheology was performed on 50% PL and control fibrin hydrogels. The PL behaved as a soft viscoelastic solid. The storage and loss modulus of the high-fibrin (2.5 mg/mL) scaffold was higher than both the PL and low fibrin (1.0 mg/mL) hydrogels (P>0.005). However, there was no difference between the 50% PL and the 1.0 mg/mL fibrin scaffolds in either the storage modulus or loss modulus (FIGS. 2E and F), despite an approximately 4-fold lower fibrin content in the PL hydrogel. To examine the release of endogenous growth factors from within the PL hydrogels, we first quantified total protein released into PBS over 20 days using a modified Bradford assay. There was no difference in the percentage of total protein released from the gels over the 20 day time course with the addition of aprotinin to the PBS (FIGS. 2G and H). To directly evaluate the release of proangiogenic growth factors from the PL scaffold, we quantified the amount of PDGF-BB released into PBS with and without aprotinin from 50% PL scaffolds over a 20-day time course using ELISA. The PL scaffold continuously released PDGF-BB for 20 days, and retained more than 45% of the of growth factor (FIGS. 2I and J). The addition of exogenous aprotinin to incubation media did not impact the growth factor release from the PL hydrogel.

### PL Scaffold Promotes Cell Sprouting in an In Vitro Angiogenesis Coculture Assay

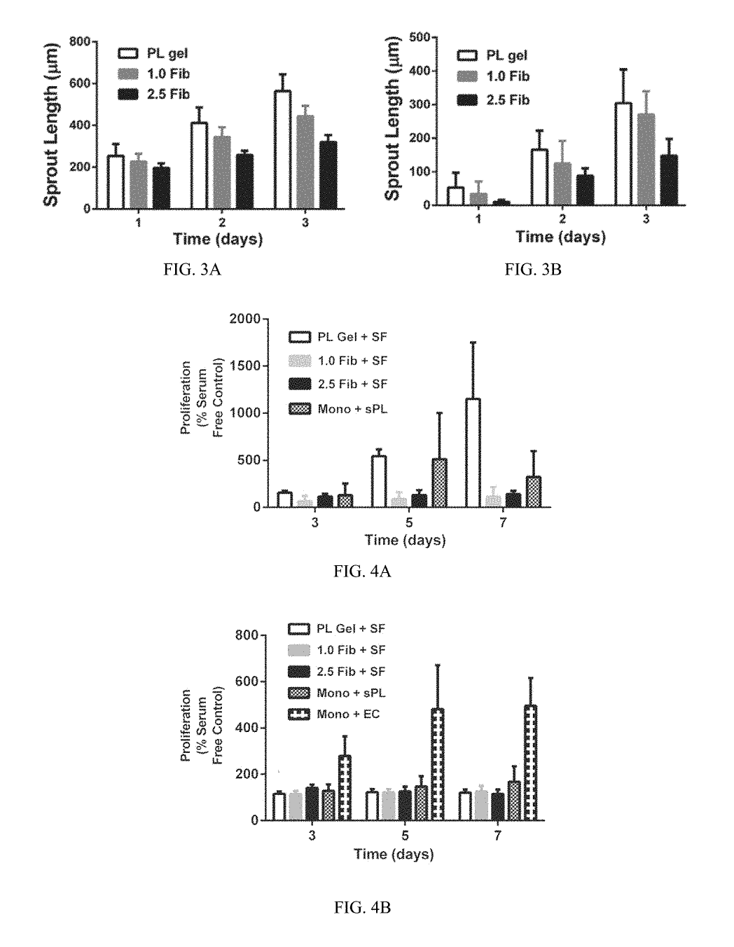

An in vitro co-culture assay was used to characterize the PL gel on MSC invasion and EC sprouting in a co-culture assay. To mimic an austere environment, MSC invasion and EC sprouting were assessed under serum free media conditions. Cell pellets containing a 1:1 ratio of human MSCs and PKH26 labeled HUVECs were embedded in 50% PL or fibrin hydrogels. Unlabeled MSC invasion was quantified under bright field microscopy and PKH26 labeled ECs were quantified under fluorescent microscopy. Both invasion (MSC) and sprout (EC) lengths were quantified over 3 days. Standard 2.5 mg/mL and a “low” concentration (1 mg/mL) fibrin gel were used as controls. Using bright field microscopy, which signified mainly MSC invasion when contrasted with fluorescent HUVECs, invasion was increased in the PL hydrogels compared to both the high and low concentration fibrin hydrogels over 3 days (FIG. 3A). Endothelial cell sprouting within the coculture assay was quantified by fluorescent imaging of the gel. At 3 days there was increased HUVEC sprouting in the PL hydrogel compared to high concentration fibrin hydrogels (FIG. 3B), but there was no measurable difference in endothelial cell sprout length between the PL gel and the low concentration fibrin group at 3 days (p=0.30).

### PL Gel Selectively Promotes MSC Proliferation

To evaluate the effect of the PL scaffold on cellular proliferation, human MSCs and HUVECs were cultured separately in PL and fibrin hydrogels under serum free conditions. Mitotic activity was measured using an MTS assay over 7 days. Growth in the PL hydrogel led to an increased proliferative response in human MSCs compared to the fibrin control hydrogels at 5 days. After 7 days, the metabolic activity of human MSCs grown in PL hydrogels under serum free conditions was significantly higher than the fibrin controls. This was also seen when MSCs grown in a monolayer were supplemented with soluble PL (FIG. 4A). There was no detectable increase in metabolic activity in HUVECs grown in PL compared to HUVECs grown in fibrin hydrogels or in a monolayer in media supplement with soluble PL (FIG. 4B). However, proliferation of HUVECs grown in a monolayer with full endothelial cell media was significantly higher than all other culture conditions (FIG. 4B), indicating that the mitogenic effect of PL preferentially stimulates MSCs over HUVECs.

PL Scaffold Promotes Invasion of both MSCs and ECs Independently

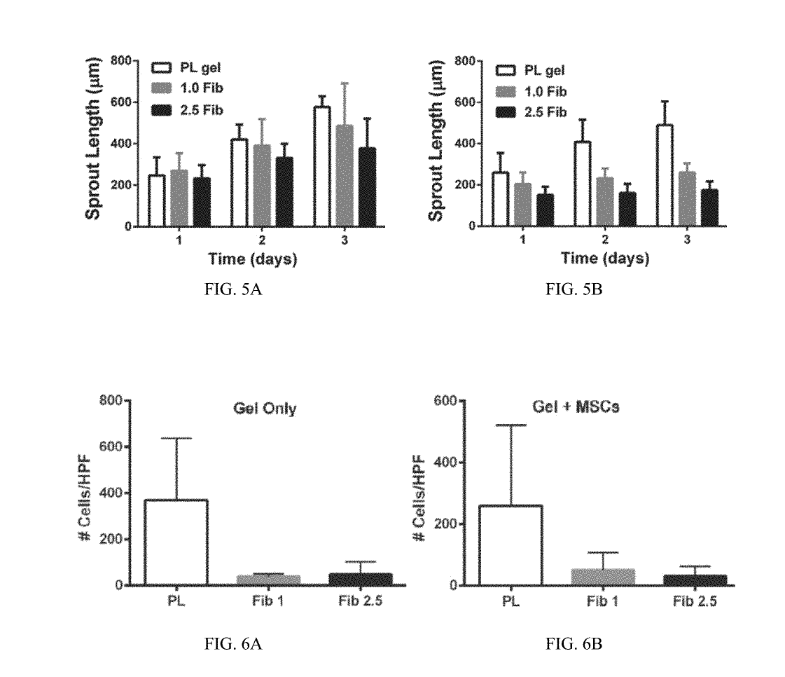

To assess the effect of the PL scaffold on MSC invasion or HUVEC sprouting in 3D, cell pellets containing only MSCs or HUVECs were embedded in PL or fibrin hydrogels, and their respective invasion/sprout length was quantified over 3 days. At the 3-day time point sprout length from MSC pellets invading the PL hydrogel was increased compared to pellets in high concentration fibrin gels with serum free media or media with soluble PL (FIG. 5A). There was no difference in sprout length between the PL and low concentration fibrin hydrogels under serum free conditions or when supplemented with soluble PL. There was also an increase in sprout length from HUVEC pellets embedded in PL compared to both fibrin hydrogels with serum free media at both 2 and 3 days (FIG. 5B). For cell therapy applications in a clinical setting, the PL would serve as a scaffold for MSC delivery, which would then recruit endogenous host endothelial cells. To assess whether the PL scaffold promotes migration of remote HUVECs toward the hydrogel, a transwell migration assay was modified so that HUVECs could migrate through a transwell insert toward the PL or a control hydrogel. HUVECs preferentially migrated toward the PL gel when compared to fibrin only gels (FIG. 6A). When MSCs were embedded within the hydrogels, the PL gel also led to a significant increase in the number of recruited HUVECs when compared to the control fibrin gels containing MSCs.

### MSCs Delivered in PL Scaffolds Lead to Rapid Neovascularization In Vivo

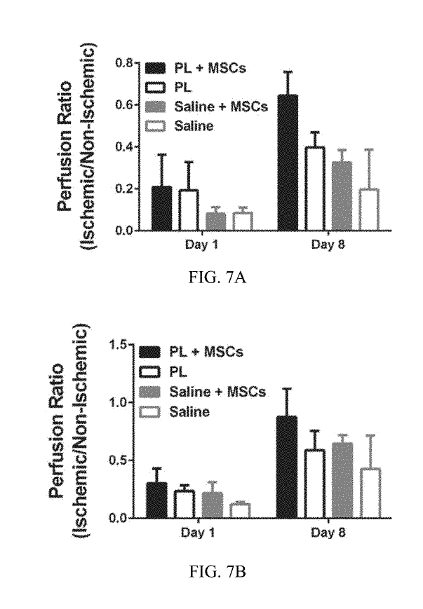

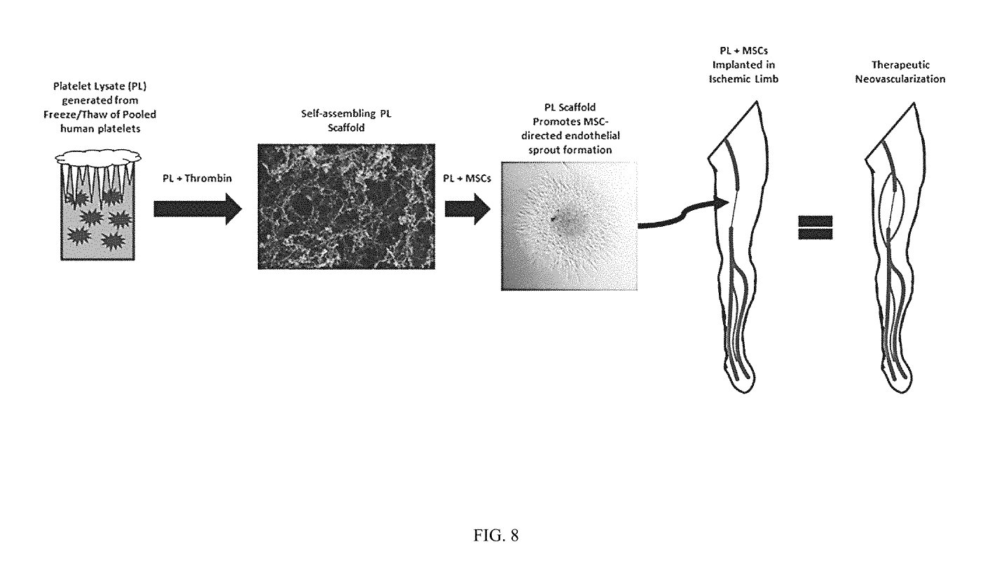

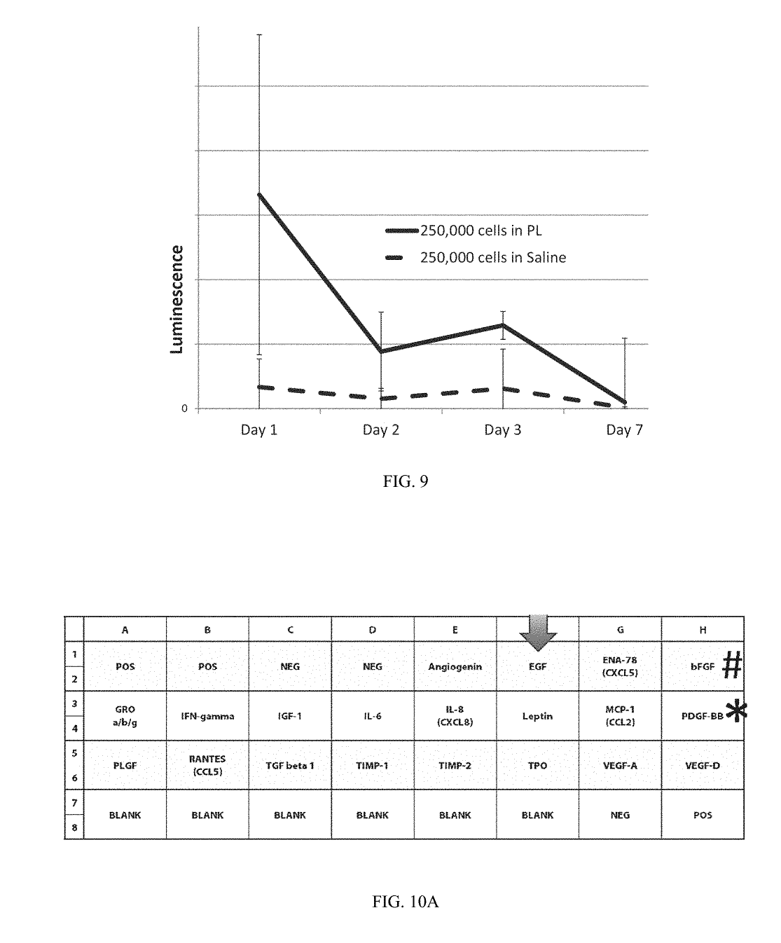

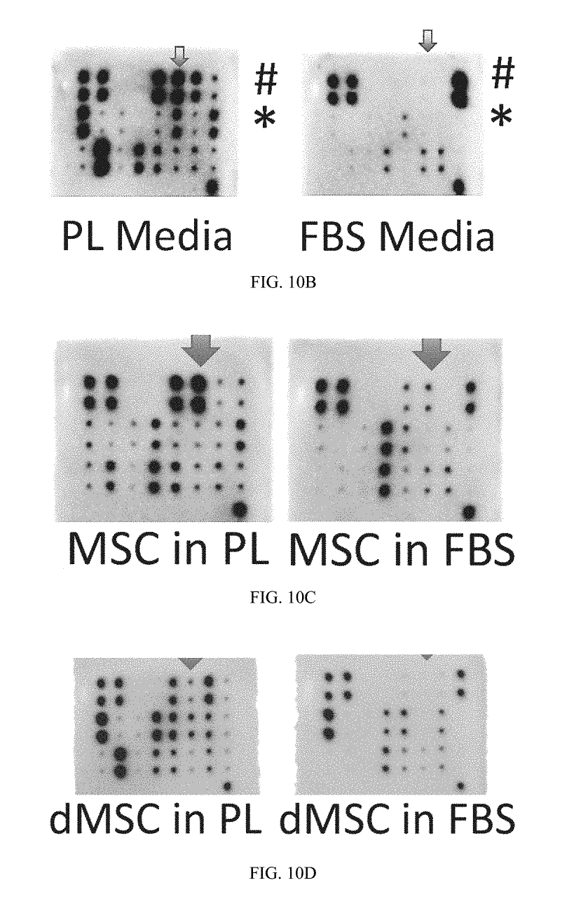

Implantation of MSCs embedded in PL into ischemic limbs in a mouse model of HLI led to rapid neovascularization of ischemic tissues by 8 days when assessed with LDPI. Perfusion ratios in the gastrocnemius muscle of mice that received PL embedded with MSCs were significantly higher than control groups containing PL only, MSCs in saline, or a saline only vehicle at 8 days (FIG. 7A). LDPI of the entire surgical leg revealed complete restoration of perfusion after 8 days (signified by a ratio of 1 compared to the contralateral limb). This was significant in comparison with the saline control groups. (FIG. 7B).

### Hydrogel Degradation

In order to test stability 0.5 mL 50% PL or fibrin hydrogels were prepared with the addition of 100 micro-gram Alexa Fluor 488 conjugated fibrinogen. Cell free hydrogels were cast in 24-well tissue culture plates and covered with PBS (with or without 10 lg/mL of aprotinin) At predetermined time points for up to 7 days the supernatant was collected off of each hydrogel and replaced with PBS. At completion of the time course, the scaffold was digested with Dispase solution (Stem Cell Technologies). The concentration of Alexa fluor 488 conjugated fibrin released in each sample was determined by comparing sample fluorescence to a standard curve using a Synergy-HT microplate leader with 485/20 Ex, 528/20 Em filter set. Three independent replicates were performed for all conditions.

### Cell Culture for Preparation of Mesenchymal Stem Cells

Human MSCs obtained through an IRB approved protocol. Human MSCs were subcultured under standard conditions with alpha-MEM (Corning) containing 1% L-glutamine (Gibco), 1% pen/strep (Gibco), and standard serum supplementation with 10% fetal bovine serum (FBS; Atlanta Biological Inc). MSCs were used at passage 5-8 for all experiments. Pooled Human Umbilical Vein Endothelial Cells (HUVECs) were obtained from Genlantis and subcultured with full endothelial cell media per the distributors recommendations. HUVECs were used at passage 3-5 for experiments.

### Proliferation Assay

Hydrogels having volume of 100 micro-L containing 2×10³ cells were cast in flat bottom tissue culture treated 96-well microplates. Hydrogels were prepared containing 50% PL, 1.0 or 2.5 mg/mL fibrinogen and either MSCs or HUVECs. Hydrogels were covered with 150 micro-L of serum free alpha-MEM. Control wells were prepared by plating 2×10³ cells per well in a monolayer in serum free media, media with 5% PL or EC media. All media conditions were supplemented with 10 micro-gram/mL of aprotinin, which is required for our fibrin gels. Samples were incubated at 37° C. for 3, 5, and 7 days. At each time point, 50 micro-L of Celltiter 96 Aqueous reagent (Promega) was added to each well and allowed to incubate for 4 h. A Synergy-HT microplate reader was used to measure absorbance at 490 nm to quantify metabolic activity. Each treatment group was normalized to MSCs or HUVECs grown in a monolayer under serum free conditions on the day the assay was performed, i.e. day 3 MSCs were normalized to MSCs grown in quiescent media for 3 days. For each group, 8 independent replicates were performed twice.

### 3-D Angiogenesis Assay

An EC sprouting assay was used to assess outgrowth of cell pellets into hydrogels. Cell pellets were created with a 1:1 ratio (total 2×10⁵ cells) of MSCs and HUVECs in wells of a 96-well suspension culture microplate preblocked with a solution of 2% HSA. Pellets were allowed to form overnight, and then embedded in 400 micro-L fibrin or PL hydrogels using a nylon ring support. To allow for positioning of the cell pellet within the fibrin hydrogel a final thrombin concentration of 0.5 U/mL was used to slow polymerization times. Hydrogels were covered with 1.0 mL of serum free alpha-MEM. In order to delineate EC invasion in co-culture, HUVECs were labeled with PKH26 (Sigma) and imaged under fluorescent microscopy. Bright field and fluorescent images were taken at 24 h intervals for 3 days using an Olympus inverted fluorescent microscope. Total cell invasion, which is predominantly MSC invasion in this assay, was quantified under bright field microscopy. EC sprouting was quantified under fluorescent imaging. To quantify sprout length, a 12-segment radial grid overlay was generated for each image, and the distance from pellet center to furthest sprout border was quantified in each segment then averaged for the image. All image analyses were performed using a custom Matlab program. In order to test the effect of PL gel on either MSCs or ECs, single cell pellets (2×10⁵ MSCs or 2×10⁵ HUVECs) were formed and tested in the same manner.

### Transwell Migration Assay

Platelet lysate or fibrin hydrogels (600 micro-L) were cast in the bottom of 24-well cell culture plates either without or embedded with 20,000 human MSCs/mL. Pre-hydrated transwell inserts (Corning) with a 6.5 micro-m thickness and 8.0 lm pore size were then placed onto the top of the gels, and 100 lL of serum free media containing 50,000 HUVECs was placed on top of the insert. After 24 h, the inserts were removed and stained with crystal violet Images were obtained of the bottom of the inserts at 10× magnification and the number of stained cells was counted. Each group was performed in quadruplicate and repeated twice. Values were reported as number of cells migrated per high power field.

### Hind Limb Ischemia Model

Twelve week old male NODSCID mice underwent right sided hind limb ischemia via ligation and removal of their femoral artery. Prior to surgery all fur was removed on bilateral hind limbs with depilatory cream. Mice were segregated into groups of PL+MSCs; P1 alone; saline+MSCs, and saline alone; N=4 in all groups. The saline vector was chosen because this is used in clinical trialing of intramuscular injections for peripheral arterial disease (PAD). Treatment volumes were 200 micro-L for all groups. There were 1,000,000 human MSCs delivered with a 23 gauge needle into each limb treated with PL+MSCs, Saline+MSCs, PL alone, or saline alone. Animals then underwent laser Doppler perfusion imaging (LDPI) on postoperative days 1 and 8. An LDPI with an 810 nm LASER (MooreLDI, Moore Instruments) was used to assess perfusion in the ischemic and nonischemic legs following arterial ligation. Scanning distance was 21 cm with a scan speed of 4 ms/pixel and a resolution of 256×256 pixels was used. Mean perfusion was quantified on each limb by setting an area of interest over the entire leg and foot, and then a separate are of interest over the gastrocnemius muscle (the ischemic portion of the leg). The results were reported as mean perfusion ration of the ischemic limb (micro-L) to the nonischemic limb (NIL).

1. A composition comprising fibrin polymer produced by mixing platelet lysate with a solution comprising minimal essential medium and thrombin.

2. The composition of claim 1, wherein the fibrin content is about 225 micro grams/mL.

3. The composition of claim 1 wherein the concentration of the calcium salt in the minimal essential medium is above 1.8 mM.

4. The composition of claim 3 wherein the concentration of the calcium salt in the minimal essential medium is about 5 mM or above.

5. The composition of claim 1, wherein the composition is about 1:1 mixture by volume of platelet lysate and the solution to produce the fibrin polymer.

6. The composition of claim 1, wherein the composition comprises human mesenchymal stem cells (MSCs) and/or adipose tissue derived stem cells (ASC).

7. The composition of claim 6, wherein the MSCs are in the form of spheroids.

8. A pharmaceutical composition comprising a fibrin polymer of claim 1.

9. The pharmaceutical composition of claim 6 in a sealed container or syringe.

10. A method of treating or preventing tissue necrosis due to ischemia comprising administering or implanting a composition of claim 1 to subject in need thereof.

11. The method of claim 10, wherein the subject is at risk of, exhibiting symptoms of, or diagnosed with peripheral arterial disease (PAD) or critical limb ischemia (CLI).

12. A method of promoting vascularization in a tissue comprising administering or implanting MSCs in the form of a spheroid.

13. The method of claim 12 wherein the spheroid is produced in a composition comprising a fibrin polymer produced by mixing platelet lysate with a solution comprising minimal essential medium and thrombin.
